(19) United States
(12) Patent Application Publication
(10) Pub. No.: US 2010/0185084 A1
(43) Pub. Date: Jul. 22, 2010

(54) Non-invasive Cardiac Characteristic Determination System

(75) Inventor: Hongxuan Zhang, Palatine, IL (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/689,331

(22) Filed: Jan. 19, 2010

Related U.S. Application Data
(60) Provisional application No. 61/146,454, filed on Jan. 22, 2009.

(51) Int. Cl. A61B 6/00; A61B 5/02; A61B 8/06

(52) U.S. Cl. 600/425; 600/526; 600/454

(57) ABSTRACT

A system uses non-invasive laser, ultrasound or electro-magnetic monitoring, to derive CO/SV, CO/SV deviation, and related cardiac function parameters. The non-invasive system determines cardiac stroke volume and includes an input processor for receiving determined values provided using a measurement processor. The determined values comprise, a blood vessel internal diameter and rate of flow of blood through the blood vessel in a heart cycle. A computation processor calculates a vessel stroke volume comprising volume of blood transferred through the blood vessel in a heart cycle using the measured blood vessel internal diameter and the rate of flow of blood. The computation processor determines cardiac stroke volume by determining a factor for use in adjusting the vessel stroke volume to provide a cardiac stroke volume and adjusting the vessel stroke volume using the determined factor to provide the cardiac stroke volume. An output processor provides data representing the determined cardiac stroke volume to a destination.

This is a non-provisional application of provisional application Ser. No. 61/146,454 filed Jan. 22, 2009, by H. Zhang.

### FIELD OF THE INVENTION

This invention concerns a non-invasive system for determining cardiac stroke volume by adjusting a calculated vessel stroke volume using a determined factor.

### BACKGROUND OF THE INVENTION

Heart stroke volume (SV) measurement and calculation are used for patient health status monitoring and qualification, especially for patients in a CCU (Critical Care Unit) and ICU (Intensive Care Unit). However known clinical methods for measuring and monitoring cardiac output (CO) and SV are typically invasive, such as by using an intra-cardiac catheter and blood pressure based signal acquisition and calculation. There are also some non-invasive and less invasive measurements for CO/SV estimation, which are based on impedance or angiographic images, for example. But these methods are usually not accurate or reliable, and need extensive expertise and clinical experience for accurate interpretation and appropriate cardiac rhythm management.

A cardiovascular system comprises, a pump (the heart), a carrier fluid (blood), a distribution network (arteries), an exchange system (capillary network) and a collecting system (venous system). Blood pressure is a driving force that propels blood along the distribution network. Stroke volume (SV) is the volume of blood pumped by the right and left ventricle of the heart in one contraction. Specifically, it is the volume of blood ejected from ventricles during systole. The stroke volume does not comprise all of the blood contained in the left ventricle. Normally, only about two-thirds of the blood in the ventricle is ejected with each beat. The blood that is actually pumped from the left ventricle comprises the stroke volume and it, together with the heart rate, determines the cardiac output (CO).

Hemodynamic and cardiac output analysis, such as SV measurement (including SV signals, SV value deviation and variation), support analysis and characterization of cardiac pathology and disorders and support prediction of life-threatening events. Hence, accurate and precise hemodynamic measurement, parameter calculation, efficient diagnosis, and reliable (True positive and false negative rate) evaluation are desirable to monitor and characterize patient health status. Additionally, known systems are typically based on invasive signal acquisition and data measurements. These systems typically need blood samples for Fick cardiac output measurement and involve subjective and inaccurate image estimation of EoD (end of diastolic) and EoS (end of systolic) points in an angiographic image for cardiac output calculation. Known systems may also involve analysis of deviation of measured data and calculations in thermodilution based cardiac output monitoring.

Known CO and SV measurement systems involving thermodilution analysis and Fick calculation, for example, are typically invasive, and inaccurate since the corresponding data acquisition is not precise because blood pressure measurements are made in a noisy environment and image acquisition is performed based on imprecise timing or gating of EoD and EoS points. Known, less-invasive or non-invasive methods for SV estimation use blood stroke volume within local vessels to proportionally estimate heart SV. The non-linear relationship between measured and actual heart SV may result in substantial calculation errors and deviation and false alarms in the monitoring, especially in critical care monitoring. In known measurement methods and approaches, invasive and non-invasive hemodynamic data acquisition, accurate calculation and diagnosis, and reliable and precise interpretation of CO/SV are usually compromised. Known methods typically involve optimization and empirical coefficients in the calculation and diagnosis. A system according to invention principles addresses these deficiencies and related problems.

### SUMMARY OF THE INVENTION

A system improves the precision and reliability of measurement and diagnosis of patient cardiac status, using non-invasive laser, ultrasound or electro-magnetic monitoring, to derive CO/SV, CO/SV deviation, and related cardiac function parameters, for example for diagnosing and quantifying patient health status. A non-invasive system determines cardiac stroke volume and includes an input processor for receiving determined values provided using a measurement processor. The determined values comprise, a blood vessel internal diameter and rate of flow of blood through the blood vessel in a heart cycle. A computation processor calculates a vessel stroke volume comprising volume of blood transferred through the blood vessel in a heart cycle using the measured blood vessel internal diameter and the rate of flow of blood. The computation processor determines cardiac stroke volume by determining a factor for use in adjusting the vessel stroke volume to provide a cardiac stroke volume and adjusting the vessel stroke volume using the determined factor to provide the cardiac stroke volume. An output processor provides data representing the determined cardiac stroke volume to a destination.

### BRIEF DESCRIPTION OF THE DRAWING

FIG. 1 shows a non-invasive system for determining cardiac stroke volume, according to invention principles.

FIG. 2 shows different positions of non-invasive methods for monitoring and analysis of hemodynamic, electrophysiology and vital sign signals, according to invention principles.

FIG. 3 shows a Table identifying stimulation and test methods for different types of signal stimulation and associated anatomical region and CO/SV measurements, according to invention principles.

FIG. 4 shows an arm artery based CO and SV test, monitoring and analysis system, according to invention principles.

FIG. 5 shows a flowchart of a process employed by a medical device for non-invasive CO and SV measurement, calculation and characterization, according to invention principles.

FIG. 6 shows an Artificial Neural Network (ANN) employing training data used in non-invasive CO and SV measurement, calculation and characterization, according to invention principles.

FIG. 7 shows a flowchart of a process used by a non-invasive system for determining cardiac stroke volume, according to invention principles.

### DETAILED DESCRIPTION OF THE INVENTION

A system improves the precision and reliability of measurement and diagnosis of patient cardiac status, using a non-invasive monitoring method based on laser, ultrasound, electro-magnetic measurement methods to derive and calculate hemodynamic, electrophysiology and vital sign signals. The non-invasive methods are used to evaluate and characterize heart CO/SV, CO/SV deviation, and related parameters for diagnosing and quantifying patient health status. The system provides a more accurate and reliable method for identifying cardiac disorders, characterizing pathological severities, predicting life-threatening events, and evaluating drug delivery effects. The system provides improved control and analysis in data acquisition and cardiac function characterization for CCU and ICU care, for example, involving processing vital sign signals in conjunction with hemodynamic signals to provide precise and stable analysis of patient health status.

The system provides a non-invasive CO/SV measurement and calculation with improved safety and may employ ultrasound, laser, microwave, electrical-magnetic, based functions applying thermodilution or Fick theory principles, for example. The system employs a combination of hemodynamic signals, electrophysiological and vital sign signals to improve precision and signal stability and avoids noise effects in measurement by using signal gating and synchronization based on an ECG (Electrocardiogram) signal, a NIBP (Non-invasive Blood Pressure) signal or SPO2 (blood oxygen saturation) signal, for example. The CO/SV measurement and calculation is performed for the heart and local circulation system. This includes calculating SV in the main vessels (especially the arteries) and calculating CO/SV in a local artery based on combination of hemodynamic, electrophysiological and vital signs and estimating heart CO/SV. In one embodiment, the system uses an ANN (Artificial Neural Network) for combining information in performing calculations and data deviation analysis, CO/SV frequency analysis, and other quantitative analysis and characterization of signal changes and distortion. The calculation and analysis is used in patient health status quantification and evaluation and in predicting life-threatening events and evaluating drug delivery effects.

Ventricular stroke volume (SV) is the difference between the ventricular end-diastolic volume (EDV) and the end-systolic volume (ESV). The EDV is the filled volume of the ventricle prior to contraction and the ESV is the residual volume of blood remaining in the ventricle after ejection. In a typical heart, the EDV is about 120 ml of blood and the ESV about 50 ml of blood. The difference in these two volumes, 70 ml, represents the SV. Therefore, a factor that alters either the EDV or the ESV changes SV.

SV=EDV−ESV, CO=HR×SV

where HR=heart rate.

Known methods for CO and SV measurement and cardiac health status index calculation include, a Fick method, a Thermodilution method, and an Angiographic image method. The Fick method requires determining oxygen consumption or VO2 and usually involves at least two blood samples (invasive). Thermodilution needs saline to be injected which flows through the RV (Right Ventricle) and cools a thermister enabling measured cooling rate and temperature to be used for CO and SV measurement and calculation (invasive). The Angiographic method is used for CO and SV calculation, and estimation of cardiac image volume at EoD and EoS time, which usually requires extensive clinical experience and may lead to substantial variation in the estimation and characterization of these values.

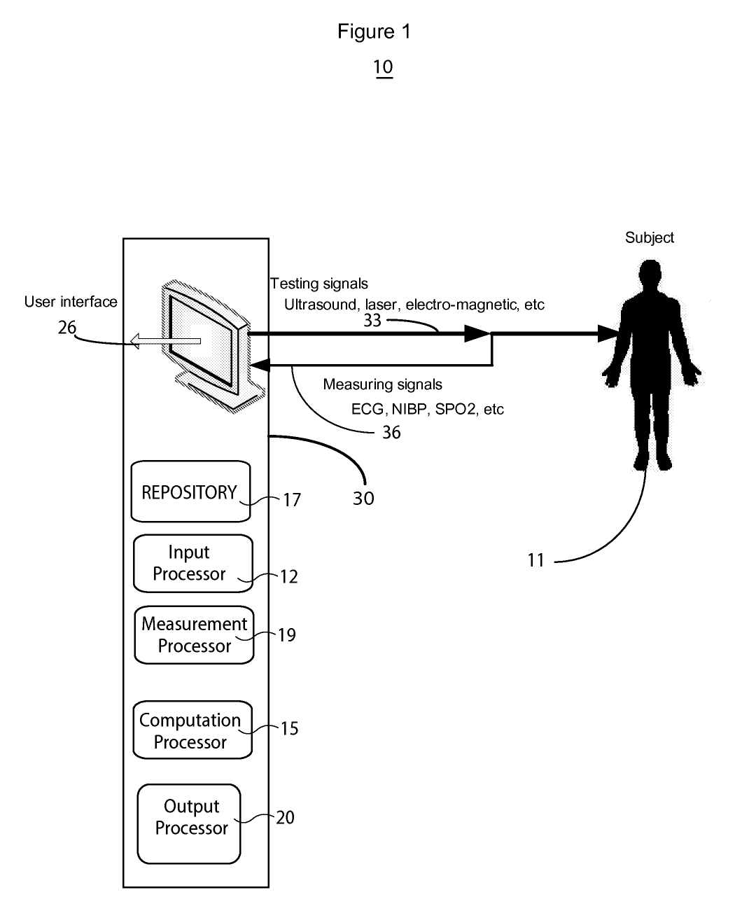

FIG. 1 shows a non-invasive system (10) for determining cardiac stroke volume. System 10 measures hemodynamic signals (such as blood flow, speed) and vital sign signals (such as NIBP, ECG, SPO2) 36 using ultrasound, microwave and/or electro-magnetic functions. FIG. 1 illustrates integration of non-invasive measurements and methods using hemodynamic (such as NIBP), electrophysiology signals (such as ECG), and vital sign signals (such as SPO2). In FIG. 1, a critical care and monitoring device 30 generates testing signals 33 for use in measurement, such as ultrasound signals and laser signals. Device 30 acquires, filters, converts and measures subject signals 36 from patient 11, such as ECG signals, NIBP signals, SPO2 signals. Device 30 calculates and displays CO/SV information and health status on a display image of user interface 26. System 10 provides integrated real time signal monitoring, recording and calculation of CO, SV, related parameters, health status, disease related patient signal distortion and prediction of life-threatening events.

System 10 performs non-invasive body parameter measurement around a heart position and local vessel position for local vessel blood SV calculation and characterization of the heart CO and SV. Critical care device 30 may be a bedside unit and portable. Non-invasive system 30 determines cardiac stroke volume using input processor 12 for receiving determined values provided using measurement processor 19. The determined values comprise, a blood vessel internal diameter and rate of flow of blood through the blood vessel in a heart cycle. Computation processor 15 calculates a vessel stroke volume comprising volume of blood transferred through the blood vessel in a heart cycle using the measured blood vessel internal diameter and the rate of flow of blood. Computation processor 15 calculates a heart stroke volume of a patient by applying a factor to a determined vessel stroke volume comprising volume of blood transferred through the blood vessel in a heart cycle using the measured blood vessel internal diameter and the rate of flow of blood. Output processor 20 provides data representing the determined cardiac stroke volume to a destination and stores it as well as a determined factor in repository 17.

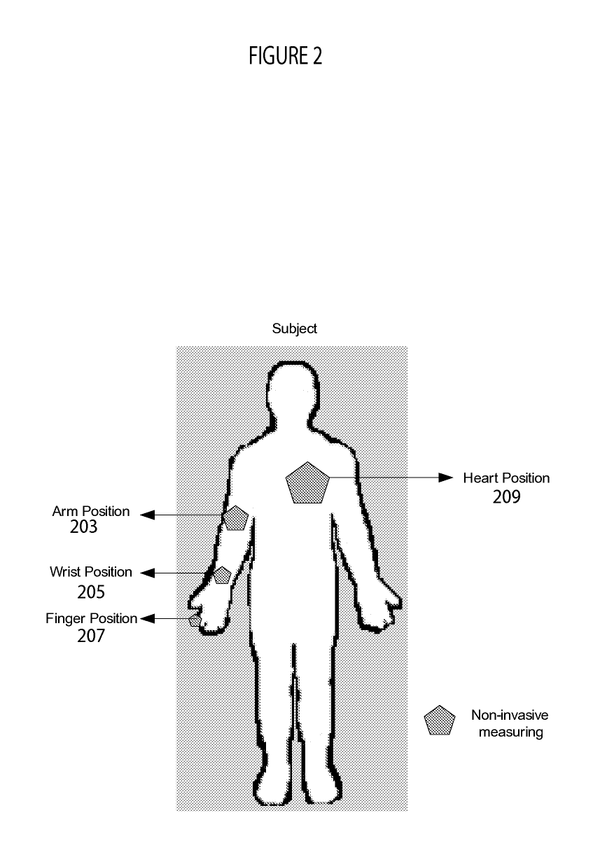

FIG. 2 shows different anatomical positions used for non-invasive CO/SV measurement methods for monitoring and analysis of hemodynamic, electrophysiology and vital sign signals. The different positions of a patient include, chest (heart) position 209, arm position 203, wrist position 205, and finger position 207 (for the capillary system). Different measurement methods are applied at the corresponding different anatomical positions for data acquisition of hemodynamic, electrophysiology and vital sign signals for CO and SV determination. The different measurement methods employ different kinds of testing signals for subject data sensing and acquisition. For example, for heart position 209, ultrasound and electromagnetic field methods are used while a laser may be used in signal acquisition and monitoring for finger position 207. Further, for wrist and arm positions 205 and 203 respectively, different kind of methods are selected based on measurement convenience, blood pressure and volume.

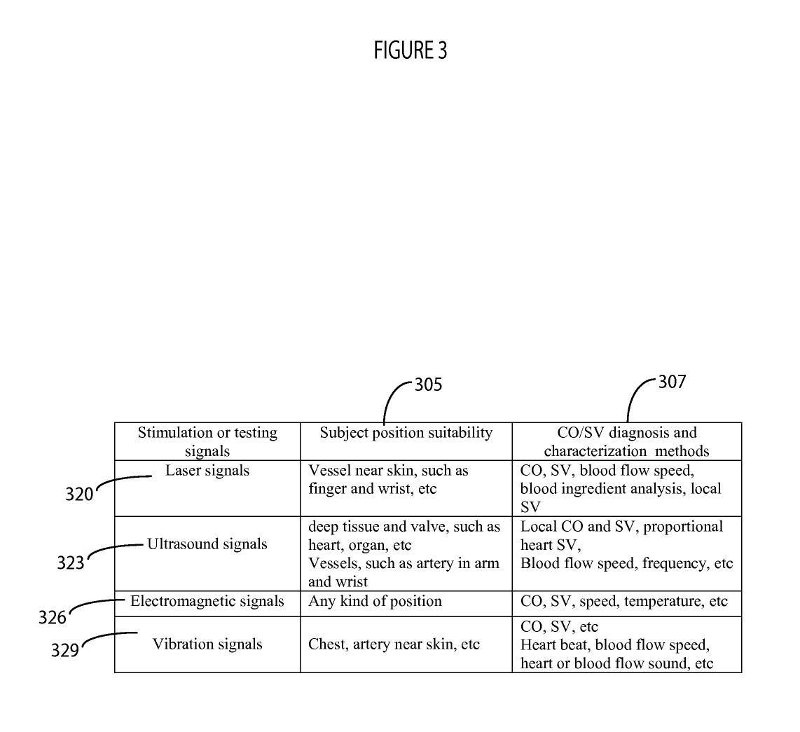

FIG. 3 shows a Table identifying stimulation and test methods for different types of signal stimulation and associated anatomical region and CO/SV measurements. Stimulation signals include laser signals 320, ultrasound signals 323, electromagnetic signals 326 and vibration signals 329. The different types of signal stimulation and test methods are applicable at different anatomical sites indicated in column 305 to make the CO/SV measurements indicated in column 307. The measurements involve different types of signal sensing, sensor, signal conversion (pressure, vibration, frequency, energy, fluid flow speed), and signal calculation.

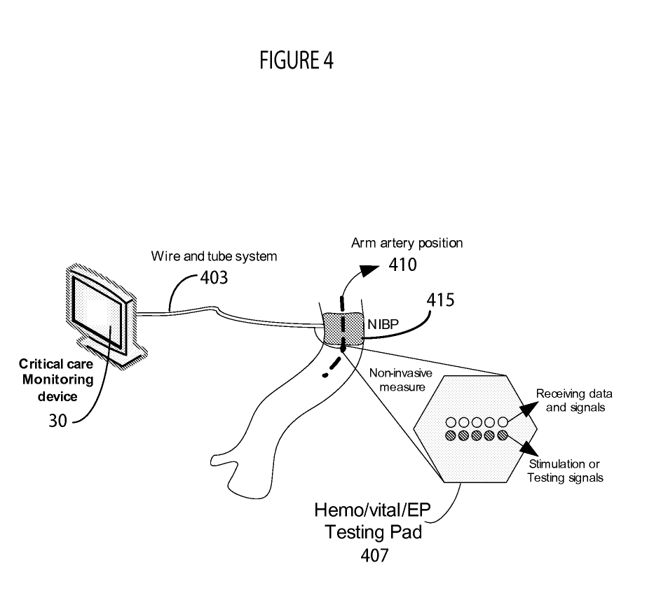

FIG. 4 shows an arm artery based CO and SV test, monitoring and analysis system. Based on the clinical situation and conditions, different kinds of sensor, measurement systems and analysis are used for convenience and optimum effectiveness in performing a medical procedure. The system employs NIBP (patient arm non-invasive blood pressure) measurement sensor 415 and a non-invasive Hemodynamic, Vital sign and Electrophysiological signal testing and measurement pad 407 attached to the patient arm specifically focused on arm artery position 410. Units 415 and 407 acquire blood signals, vibration signals and other related information directly and precisely for processing by critical care monitoring device 30. Specifically units 407 and 415 acquire hemodynamic signals, vital sign signals and electrophysiological signals (such as blood pressure, blood flow, SPO2, ECG). Stimulation signals (such as laser and ultrasound stimulation signals) are generated by critical care monitoring device 30 and provided to arm artery position 410. Units 407 and 415 also acquire corresponding patient signals, including blood flow speed, sound and instantaneous frequency, for example, at the arm artery position 410. Thereby, device 30 derives CO/SV related signals.

Artery vessel size and diameter at position 410 are precisely measured. Device 30 calculates and characterizes CO/SV and related health parameters using,

CO/SV=ƒ (speed, size, frequency)

where, ƒ(•) is a function used for CO and SV calculation; speed is the blood flow speed in the vessel, size is the size (e.g., diameter, cross-sectional area) parameter of the vessel, frequency is the instantaneous frequency which can be utilized for tuning of blood flow speed deviation. In other embodiments other parameters for the function ƒ(•) are used to calculate the CO and SV of the local artery. The heart CO/SV can be matched and characterized by mathematical methods, such as using an ANN (artificial neural network). Usually the relationship between local artery CO/SV and heart CO/SV is nonlinear and an ANN facilitates nonlinear calculation and quantification. Testing pad 407 supports 10 leads for stimulation and testing signals and receives data, but in other embodiments the number of leads is variable in response to clinical application and situation. The method is not limited to CO and SV determination based on arm arteries but is applicable to the heart (using body surface), wrist and finger for signal acquisition, data calculation and health status characterization.

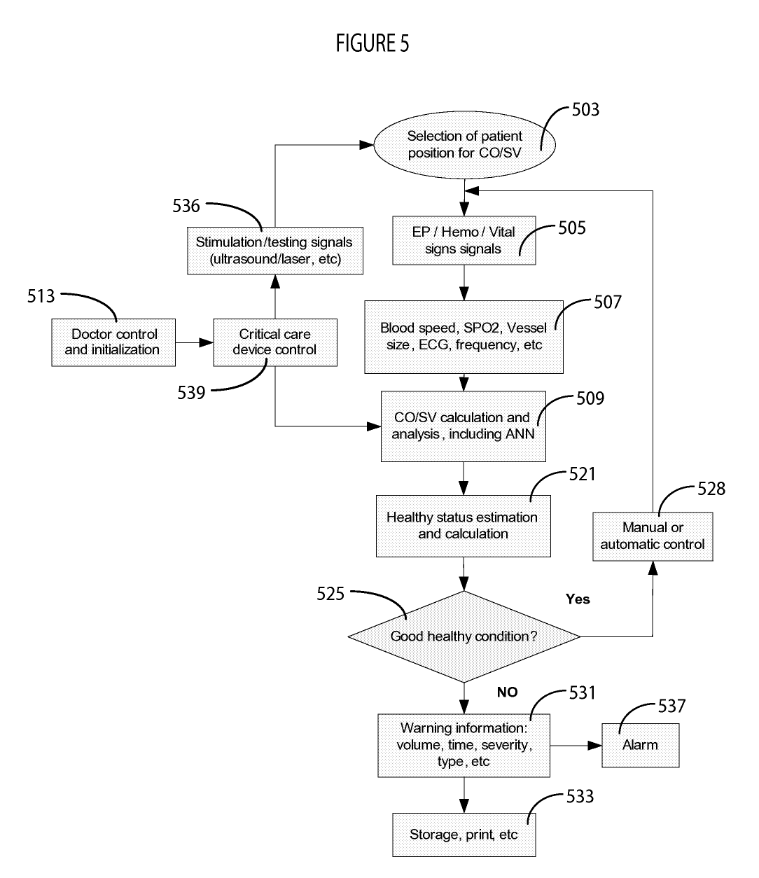

FIG. 5 shows a flowchart of a process employed by a medical device for non-invasive CO and SV measurement, calculation and characterization. Critical care monitoring device 30 (FIG. 1) controls stimulation signal generation (of ultrasound, laser or electro-magnetic signals), used to measure patient parameters, such as blood speed, ECG, blood sound, blood flow frequency, size and diameter of the artery vessel. Device 30 uses the parameters to calculate and characterize CO, SV and health status index, such as pathology severity and time. For example, ultrasound is used to precisely measure blood flow speed and size of vessel. A laser signal is used to measure blood content and ingredients by using different laser wavelength. The system in response to SPO2 and ECG signals, excludes noisy and distorted signal portions to enhance precision of non-invasive measurement and calculation. System 10 determines time of occurrence of cardiac events and severity and type of cardiac event by calculating CO/SV and a health index.

Critical care monitoring device 30 (FIG. 1) selects an anatomical position and corresponding type of stimulation signal generator (laser, electromagnetic, vibration or ultrasound) for use in determining CO/SV in step 503. Device 30 acquires hemodynamic, vital sign and electrophysiological signals over the duration of one or more heart cycles in step 505 and measures parameters in step 507 using the selected type of stimulation signal generator. The measured parameters comprise blood speed and frequency, SPO2, vessel size and ECG, for example. Device 30 records and processes the patient hemodynamic signals, electrophysiological signals, and vital signs, to derive other parameter values. In step 509, device 30 performs CO/SV calculation and analysis. Device 30 uses ECG signal gating to reduce patient noise and electrical noise and accommodate arrhythmia effects in CO and SV calculation and derives a precise integration time. It is assumed that the cardiac blood flow speed is a function (Φ(t)) of time and blood vessel dimension (size and diameter) is also a time varying function (θ(t)). The blood SV is calculated using following equation:

$$S V = {\int }_{t ∈ T} {π ( \frac{θ ( t ) d t}{2} )}^{2} Φ ( t ) d t$$

where T is the RR wave time that is derived from an ECG signal.

In one embodiment, device 30 employs an ANN (Artificial Neural Network) unit to calculate and characterize heart CO and SV by determining a nonlinear relationship between a local vessel and a heart for use in diagnosis and analysis.

In step 521, device 30 derives a health index value based on calculated CO and SV parameters as well as other patient parameters. In step 525 device 30 identifies a particular medical condition by mapping determined CO and SV parameters as well as the other patient parameters and calculated values to corresponding value ranges associated with medical conditions using predetermined mapping information stored in repository 17. Upon a determination of good health condition in step 525, device 30 in step 528 in response to user manual command or automatic executable application command, re-iterates performance of steps 505, 507, 509 and 521 involving re-acquiring hemodynamic, vital sign and electrophysiological signals and computing CO and SV parameters and health status as a continuous monitoring process or as an intermittent user initiated process. In response to a determination of impaired health condition in step 525, device 30 in step 531 determines characteristics including, location, size, volume, severity and type of medical condition as well as a time within a heart cycle associated with a medical condition. Device 30 initiates generation of an alert message indicating the determined characteristics for communication to a user in step 537 and provides medical information for use by a physician in making treatment decisions. Device 30 in step 533 presents calculated data and medical condition information to a user on a reproduction device such as a display or a printer and stores the data in repository 17 and prompts a user with mapped treatment suggestions.

Critical care monitoring device 30 in step 539 controls performance of CO and SV calculation (performed in step 509) and via step 536 controls selection of an anatomical position and corresponding type of stimulation signal generator (laser, electromagnetic, vibration or ultrasound) performed in step 503. Critical care monitoring device 30 performs control functions in step 539 in response to predetermined selected configuration data of a physician or configuration data associated with a particular clinical procedure and data indicating a type of clinical procedure and/or user entered data and commands provided in step 513.

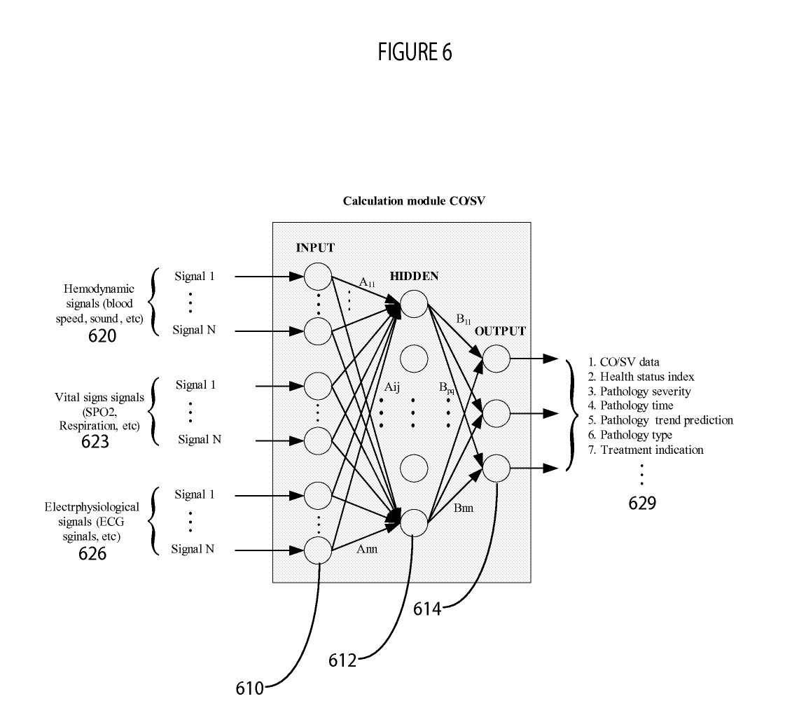

FIG. 6 shows Artificial Neural Network (ANN) 607 employing training data used in non-invasive heart CO and SV measurement, calculation and characterization. ANN 607 estimates heart CO and SV using CO and SV parameter values calculated for a local artery by determining a nonlinear relationship between heart and local artery CO/SV values using training data processed by ANN 607.

ANN unit 607 maps hemodynamic signals 620, vital sign signals 623 and electrophysiological signals 626 including calculated vessel SV and CO to heart CO and SV values, a patient health status index, pathology severity indicator, a time of a cardiac event, a pathology trend indication, a pathology type indication and candidate treatment suggestions, 629. ANN unit 607 structure comprises 3 layers, an input layer 610, hidden layer 612 and output layer 614. ANN unit Aᵢⱼ weights are applied between input layer 610 and hidden layer 612 components of the ANN computation and B_{pq} weights are applied between hidden layer 612 and calculation index components 614 of the ANN computation. The Aᵢⱼ weights and B_{pq} weights are adaptively adjusted and tuned using a training data set. ANN unit 607 incorporates a self-learning function that processes signals 620, 623 and 626 to increase the accuracy and precision of calculated results. ANN unit 607 analyzes input signal ratios by performing pattern analysis to identify pertinent ratio patterns in a heart chamber, for example, and mapping determined CO and SV parameters to a candidate diagnosis or treatment suggestion to localize a tissue impairment within an organ and determine time of occurrence within a heart cycle. ANN unit 607 also identifies arrhythmia type (e.g., AF, MI, VT, VF), severity of arrhythmia treatment and urgency level and is usable for automatic heart condition detection, diagnosis, warning and treatment. Further unit 607 performs statistical analysis to construct a threshold used to detect tissue impairment and diagnose and predict cardiac arrhythmia and pathology.

Following a training phase with a training data set, ANN unit 607 maps signals 620, 623 and 626 to data indicating an Arrhythmia type, Arrhythmia severity, candidate treatment suggestions, localized tissue impairment information identifying the cardiac arrhythmia position, pathology conducting sequence, abnormal tissue area and focus of the disorder and irregularity, for example. System 10 analyzes cardiac electrophysiological signals (including ECG and internal cardiac electrograms) based on predetermined mapping information to identify cardiac disorders, differentiate cardiac arrhythmias and quantitative and qualitative analysis and characterization of cardiac pathology and events. The severity threshold of a pathology mapping decision may vary from person to person and is adjusted at the beginning of analysis and in one embodiment may be dynamically adjusted in response to a signal quality or noise measurement, for example. The system may be advantageously utilized in general patient monitoring, implantable cardiac devices for real time automatic analysis and detection of cardiac arrhythmias and abnormalities.

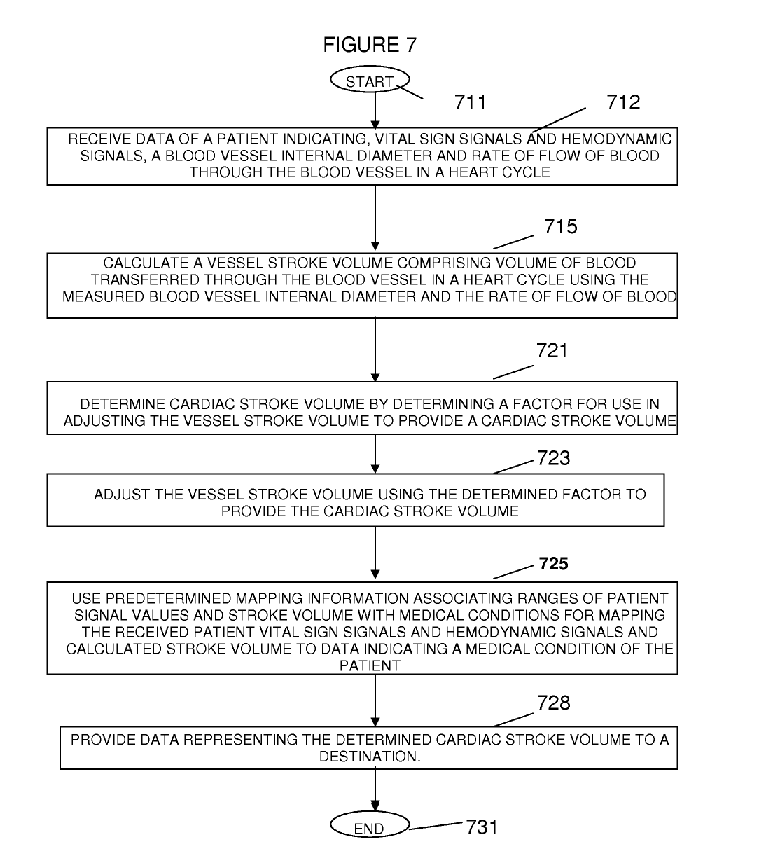

FIG. 7 shows a flowchart of a process used by non-invasive system 10 for determining cardiac stroke volume. Input processor 12 in step 712 following the start at step 711, receives data indicating, vital sign signals and hemodynamic signals of a patient, a blood vessel internal diameter and rate of flow of blood through the blood vessel in a heart cycle provided by measurement processor 19. Measurement processor 19 non-invasively measures the blood vessel internal diameter and rate of flow of blood through the blood vessel in a heart cycle using, an ultrasound imaging system, a laser based measurement system or a microwave based measurement system adaptively selected based on anatomical location of a selected artery. Specifically, measurement processor 19 provides the determined values in response to selection of the vessel in a location including at least one of, (a) a limb, (b) a wrist, (c) a finger and (d) a heart.

In step 715, computation processor 15 calculates a vessel stroke volume comprising volume of blood transferred through the blood vessel in a heart cycle using the measured blood vessel internal diameter and the rate of flow of blood. In step 721, computation processor 15 determines cardiac stroke volume by determining a factor for use in adjusting the vessel stroke volume to provide a cardiac stroke volume and in step 723 adjusts the vessel stroke volume using the determined factor to provide the cardiac stroke volume. Computation processor 15 determines the factor from a patient specific look-up table including factor representative data associated with one or more particular patient blood vessels derived for the patient concerned from stroke volume measurements performed on prior occasions.

In another embodiment, computation processor 15 determines the factor from a look-up table including factor representative data associated with at least two of, blood vessel diameter, rate of flow of blood through a blood vessel, patient height, patient weight, patient gender, patient race, patient age and patient pregnancy status and location of a vessel in a body. Computation processor 15 determines the factor using an artificial neural network configured using a training data set comprising data for the patient concerned. In one embodiment, the artificial neural network is configured using a training data set selected from a plurality of training data sets using demographic data of the patient concerned comprising one or more of, age, height, weight, gender and pregnancy status. Computation processor 15 calculates a cardiac output (CO) value using the determined cardiac stroke volume.

A mapping processor in unit 15 in step 725 uses predetermined mapping information in repository 17 associating ranges of patient signal values and stroke volume with medical conditions for mapping the received patient vital sign signals and hemodynamic signals and calculated stroke volume to data indicating a medical condition of the patient. In step 728 output processor 20 provides data representing the determined cardiac stroke volume to a destination. The process of FIG. 7 terminates at step 731.

A processor as used herein is a device for executing stored machine-readable instructions for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example. A processor may be electrically coupled with any other processor enabling interaction and/or communication there-between. A processor comprising executable instructions may be electrically coupled by being within stored executable instruction enabling interaction and/or communication with executable instructions comprising another processor. A user interface processor or generator is a known element comprising electronic circuitry or software or a combination of both for generating display images or portions thereof. A user interface comprises one or more display images enabling user interaction with a processor or other device.

An executable application comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a user interface processor and enabling user interaction with a processor or other device and associated data acquisition and processing functions.

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

The system and processes of FIGS. 1-7 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The system computes a heart stroke volume of a patient by applying a factor to a determined vessel stroke volume and the vessel stroke volume is determined as volume of blood transferred through the blood vessel in a heart cycle using a measured blood vessel internal diameter and a rate of flow of blood. Further, the processes and applications may, in alternative embodiments, be located on one or more (e.g., distributed) processing devices on a network connecting the elements of FIG. 1. Any of the functions and steps provided in FIGS. 1-7 may be implemented in hardware, software or a combination of both.

What is claimed is:

1. A non-invasive system for determining cardiac stroke volume, comprising:
an input processor for receiving vital sign signals and hemodynamic signals of a patient,
a computation processor for calculating a heart stroke volume of said patient by applying a factor to a determined vessel stroke volume, said vessel stroke volume being determined as volume of blood transferred through the blood vessel in a heart cycle using the measured blood vessel internal diameter and the rate of flow of blood;
a mapping processor for using predetermined mapping information associating ranges of patient signal values and stroke volume with medical conditions for mapping the received patient vital sign signals and hemodynamic signals and calculated stroke volume to data indicating a medical condition of said patient; and
an output processor for providing data representing the indicated medical condition to a destination device.

2. A system according to claim 1, wherein
said computation processor non-invasively determines said vessel stroke volume by measuring said blood vessel internal diameter and rate of flow of blood through said blood vessel in a heart cycle using an ultrasound imaging system.

3. A non-invasive system for determining cardiac stroke volume, comprising:
an input processor for receiving determined values provided using a measurement processor, said determined values comprising,
a blood vessel internal diameter and
rate of flow of blood through said blood vessel in a heart cycle;
a computation processor for,
calculating a vessel stroke volume comprising volume of blood transferred through the blood vessel in a heart cycle using the measured blood vessel internal diameter and the rate of flow of blood,
calculating cardiac stroke volume by determining a factor for use in adjusting said vessel stroke volume to provide a cardiac stroke volume and
adjusting said vessel stroke volume using the determined factor to provide said cardiac stroke volume; and
an output processor for providing data representing the determined cardiac stroke volume to a destination.

4. A system according to claim 3, wherein
said measurement processor non-invasively measures said blood vessel internal diameter and rate of flow of blood through said blood vessel in a heart cycle using an ultrasound imaging system.

5. A system according to claim 3, wherein
said measurement processor non-invasively measures said blood vessel internal diameter and rate of flow of blood through said blood vessel in a heart cycle using at least one of, (a) a laser based measurement system and (b) a microwave based measurement system.

6. A system according to claim 3, wherein
said computation processor determines said factor from a patient specific look-up table including factor representative data associated with one or more particular patient blood vessels derived for the patient concerned from stroke volume measurements performed on prior occasions.

7. A system according to claim 3, wherein
said factor representative data is derived for the patient concerned using prior invasive stroke volume measurements performed on prior occasions.

8. A system according to claim 3, wherein
said computation processor determines said factor using an artificial neural network.

9. A system according to claim 8, wherein
said artificial neural network is configured using a training data set comprising data for the patient concerned.

10. A system according to claim 8, wherein
said artificial neural network is configured using a training data set selected from a plurality of training data sets using demographic data of the patient concerned.

11. A system according to claim 8, wherein
said demographic data comprises at least two of, age, height, weight, gender and pregnancy status.

12. A system according to claim 3, wherein
said computation processor calculates a cardiac output (CO) value using the determined cardiac stroke volume.

13. A system according to claim 3, wherein
said computation processor determines said factor from a look-up table including factor representative data associated with at least two of, blood vessel diameter, rate of flow of blood through a blood vessel, patient height, patient weight, patient gender, patient race, patient age and patient pregnancy status, location of a vessel in a body.

14. A system according to claim 3, wherein
said measurement processor provides said determined values in response to selection of the vessel in a location including at least one of, (a) a limb, (b) a wrist, (c) a finger and (d) a heart.

15. A non-invasive method for determining cardiac stroke volume, comprising the activities of:
receiving data indicating,
a blood vessel internal diameter and
rate of flow of blood through said blood vessel in a heart cycle;
calculating a vessel stroke volume comprising volume of blood transferred through the blood vessel in a heart cycle using the measured blood vessel internal diameter and the rate of flow of blood,
determining cardiac stroke volume by determining a factor for use in adjusting said vessel stroke volume to provide a cardiac stroke volume and
adjusting said vessel stroke volume using the determined factor to provide said cardiac stroke volume; and
providing data representing the determined cardiac stroke volume to a destination.
